(19) United States
(12) Patent Application Publication
(10) Pub. No.: US 2014/0358105 A1
(43) Pub. Date: Dec. 4, 2014

(54) PEG-SHAPED OR CYLINDRICAL HYGIENE ELEMENT

(71) Applicant: Andreas Schmidt, Duderstadt (DE)

(72) Inventor: Andreas Schmidt, Duderstadt (DE)

(73) Assignee: MCAIRLAIDS VLIESSTOFFE GMBH, BERLINGERODE (DE)

(21) Appl. No.: 14/362,715

(22) PCT Filed: Dec. 5, 2012
(86) PCT No.: PCT/DE2012/100369

§ 371 Date: Jun. 4, 2014

(30) Foreign Application Priority Data
Dec. 7, 2011 (DE) 20 2011 052 225.3

(51) Int. Cl. A61F 13/20

(52) U.S. Cl. CPC A61F 13/206; A61F 13/2065
USPC 604/375

(57) ABSTRACT

A peg-shaped or cylindrical hygiene article for insertion into a body cavity or opening is claimed, which article comprises a fibrous layer (2) of pulp fibers which are interconnected in some regions by means of pressing, with the pulp fibers being compacted in the pressed regions (4), wherein the fibrous layer (2) is in the form of a fibrous web that is folded so as to form channels (11) extending in the longitudinal direction of the hygiene article.

The present invention relates to a peg-shaped or cylindrical hygiene article for insertion into a body cavity or opening, and to the use of this hygiene article as a plug for taking up body fluids.

Plugging material is used in medicine to fill natural or artificial cavities or openings. It generally serves to take up and bind blood or other body fluids, e.g. as drainage. Plugging material is often referred to in the hygiene sector as a tampon. This is generally a cotton wool or gauze swab which can also be cylindrical or peg-shaped depending on the intended use. Plugs are usually produced from cellulose materials, e.g. from cotton or viscose fibers. Although the plugs known from the prior art have good absorbency for body fluids, they also fasten or even adhere to the surface of the body cavity or opening on which they lie. Therefore, removal of the plug may in the worst case cause damage to this skin surface, or at least cause discomfort for the patient.

The object of the present invention was therefore to make available a hygiene article that is suitable for taking up and retaining body fluids from a body cavity or opening, without the disadvantages of the absorbent body known for such purposes from the prior art.

The subject matter of the present invention is accordingly a cylindrical or peg-shaped hygiene article for insertion into a body cavity or opening, which article comprises a fibrous layer of pulp fibers which are interconnected in some regions by means of pressing, with the pulp fibers being compacted in the pressed regions, wherein the fibrous layer is in the form of a fibrous web that is folded so as to form channels extending in the longitudinal direction of the hygiene article.

The nature and design of the hygiene article according to the invention permit a rapid uptake of the body fluid, without the article fastening to the surface of the body cavity or opening or even adhering thereto.

The hygiene article according to the invention is cylindrical or peg-shaped. The cross section can be round, square, oval or elliptic, preference being given to a round, oval or elliptic cross section.

In one possible embodiment, the jacket surface and possibly also one or both of the end faces of the hygiene article according to the invention are provided at least partially with a liquid-permeable sheath. The sheath can be a nonwoven material, a net, a tissue, a foil, a structured foil or a polymer film. The sheath is intended to be designed such that adhesion of the hygiene article to the body surface is largely avoided, yet the sheath has a certain porosity and permeability to the body fluid.

The hygiene article according to the invention substantially comprises a fibrous layer of pulp fibers, which layer is present in the form of a fibrous web. The pulp fibers are an essential constituent, they represent a natural material, cellulose or pulp, which is present substantially in the form of irregularly arranged fibers. Cellulose fibers in the form of nonwovens, in particular as airlaid nonwovens, are preferably used. Nonwovens are among the textile composites, they are flexible, porous sheet materials produced by interlacing and/or cohesive and/or adhesive connection of fibers. The nonwovens are loose materials of fibers which are held together generally by the inherent adherence of the fibers. The nonwovens can be strengthened if necessary. For the use in the present invention, mechanical strengthening without a binder is preferred.

In one possible embodiment of the present invention, the fibrous layer of compacted pulp fibers has a further layer on at least one flat side. It is also possible for the material to have further layers on both sides, which further layers can be of the same materials or of materials different from each other. For example, the materials for these further layers can be tissue, textile, nonwoven and/or foil-like materials. Preferably, the material for the further layer or for the further layers is likewise composed of cellulose and an easily biodegradable synthetic material. The further layers increase the strength of the fibrous layer and thus facilitate the industrial processing in high-speed machinery.

According to the invention, the fibrous layer of pulp fibers are interconnected in some regions by means of pressing, with the pulp fibers being compacted in the pressed regions. In a preferred embodiment of the present invention, it is proposed that the pulp fibers and the optionally present further layer(s) are interconnected in the pressed regions by exposure to heat. In one possible embodiment, the fibrous layer and the further layer are likewise interconnected by pressing only in some regions by means of exposure to heat. The production of such interconnected pulp fibers and of fibrous layers or webs is described, for example, in the German patent applications DE 198 24 825, DE 198 03 837, DE 197 50 890 and in the European patent EP 1 032 342.

It was found that, by using the pulp fibers pressed in only some regions as an absorption material, it is possible to achieve a very good absorbency and also a retention of the body fluids to be taken up. It is assumed that this advantageous effect is achieved in the particularly advantageous surface structure that is obtained by the pressing on the flat sides of the fibrous layer. The pressed regions give the fibrous layer a degree of strength, while the unpressed regions, in which the fibers form only a loose union, ensure a good absorbency and retention effect.

In a further possible embodiment, the surface structure of the fibrous layer can be varied by folding and/or crimping the planar fibrous layer in such a way that it acquires a crimped or undulated appearance. The extent of the planar structure is then shorter in the direction of folding/crimping. This crimping or folding can be regular or irregular. This kind of structural modification can also be referred to as creping or gathering.

According to the invention, the fibrous layer is folded so as to form channels extending in the longitudinal direction of the hygiene article. The longitudinally extending channels can be obtained, for example, by the fibrous layer, i.e. the fibrous web, being wound and/or arranged in alternating folds. For the use for taking up body fluids, it is possible in one such embodiment for the liquid to pass into the filter material via one or both end faces of the hygiene article, to be taken up via the longitudinally extending channels and to be absorbed by the fibrous layer. It is also possible for the liquid to be taken up in a direction transverse to the channels (absorbency of the fibrous layer as such).

The size (the diameter) of the channels formed can be adjusted on the basis of the geometry of the folding of the fibrous layer. The thinner the layer of fibrous material, the easier it is to adjust the size of these channels. On the other hand, the thickness of the pulp fibers that are only partially pressed must not be too small, since otherwise the strength of the fibrous web is not sufficient to be processed into a peg-shaped hygiene article. The layer thickness of the fibrous web is preferably from 0.3 to 1.8 mm.

The fibrous layer or fibrous web used according to the invention normally has a weight per unit area of between 15 g/m² and 600 g/m², preferably between 20 g/m² and 200 g/m². The diameter of the hygiene article is preferably between 2 and 200 mm, with sizes of 3 to 15 mm preferably being used.

The absorbency of the hygiene article according to the invention can be increased by additives. These additives can be, for example, absorbents or also fragrances, flavorings, aromas, pharmaceutically active substances or further auxiliaries, such as superabsorbent polymers, activated carbons, zeolites, silicates, SiO₂, disinfecting or hemostatic substances, surface-active anesthetics or analgesics, menthol, clove oil, cocoa, vanilla or any desired mixtures of the above. These substances can be applied to the sheath or can be incorporated into the article. It is also possible to impregnate the article with solutions slurries of these substances.

The absorbents that may optionally be included are distinguished by a large surface area and/or a high degree of absorbency for the body fluids and the constituents of the latter. The superabsorbent polymers have a particularly high degree of absorbency. They can be chosen from, for example, polymers based on carboxymethylcellulose derivatives or polymers based on (meth)acrylic acid and/or (meth)acrylates, and cyclodextrins. Further additives can be auxiliaries and fillers, such as titanium dioxide, chalk or kaolin, or plant substances such as flavorings and fragrances.

Pharmaceutically active substances are included particularly if the hygiene article is used in the context of a medical treatment. Preferably used active substances are disinfecting or hemostatic substances and/or surface-active anesthetics and analgesics.

Particularly for use of the hygiene article in the oral cavity, it can contain fragrances, flavorings and aromas and also further additives that improve the taste of the hygiene article. These include menthol, clove oil, cocoa, vanilla and comparable substances. Some of these substances also have antiseptic and antimicrobial effects such that, particularly for use in the oral cavity, a certain degree of disinfection can be achieved.

The auxiliaries and additives can be added by being scattered or sprayed on to the fibrous layer, e.g. onto the fibers, before or after the pressing, or else by direct injection into the peg-shaped hygiene article.

For the use as a peg-shaped hygiene article, the fibrous material, generally present as a sheet, can be brought into a preferably cylindrical shape in a manner known per se.

In one possible embodiment, the fibrous layer present in the form of a fibrous web is surrounded by a jacket layer or sheath. This jacket layer or sheath can serve to maintain the shape of the peg-shaped hygiene article. The jacket layer is preferably chosen from such a material that adhesion of the hygiene article to the surface of the body cavity or opening is substantially avoided and, even when a large part of the body fluid has been taken up, the hygiene article can be removed from the body cavity or opening without any problem. The jacket layer preferably has openings through which the body fluid can pass into the hygiene article. In this way, the absorbency can be further optimized.

The invention is set out in more detail on the basis of an illustrative embodiment and with reference to the attached figures, in which:

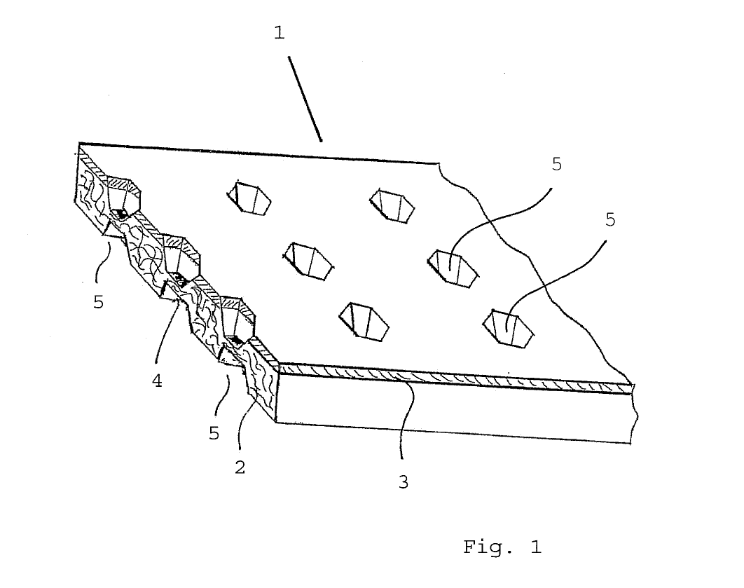

FIG. 1 shows a perspective view of the fibrous web with two layers used to produce the hygiene article,

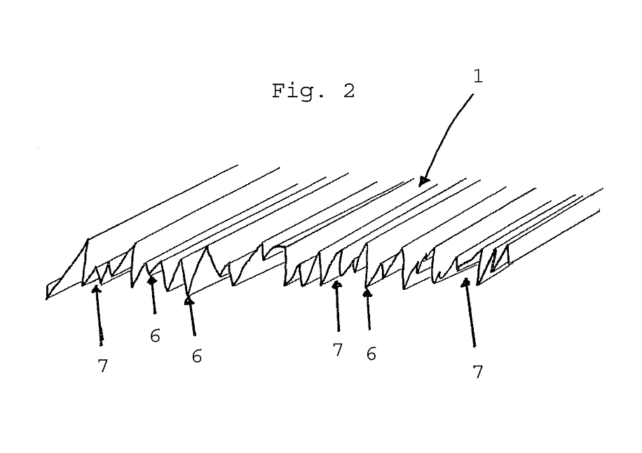

FIG. 2 shows a perspective cross section through the fibrous web in the crimped state,

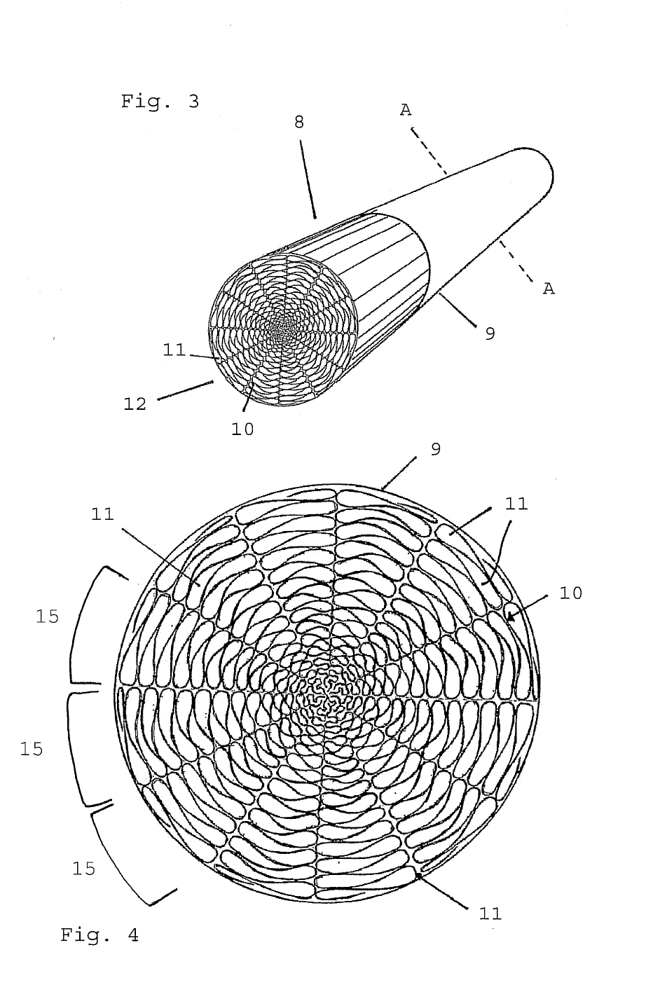

FIG. 3 shows a perspective view of the finished hygiene article, with partial omission of the jacket layer,

FIG. 4 shows a cross section through the hygiene article along the line A-A in FIG. 3.

FIG. 1 is an enlarged perspective view showing the fibrous web used in the hygiene article according to the invention. The thickness shown has been exaggerated. In the embodiment shown here, the fibrous web 1 comprises two layers, namely a fibrous layer 2 and what is by comparison a thinner further layer 3.

The lower, thicker layer is a fibrous layer 2 of pulp fibers, optionally with additives. This layer 2 is preferably produced from cellulose material in an airlaid method. Cellulose material of this kind is available economically as a bulk material. During production in a continuous airlaid method, the fibrous web is produced from a loose filling of pulp fibers (fluff pulp) layered on in an air stream and, optionally, from absorber materials and further additives and auxiliaries scattered in. For the production of a standardized product, recourse can be had to the renewable wood raw materials available on the market.

The process of loose pulp filling in an air stream as a starting product for the fibrous layer 2 permits dry processing of the pulp fibers and therefore, during the subsequent connection to the further layer, a good compaction of the pulp fibers in individual, i.e. discrete, embossed regions 4. Outside these embossed regions 4, the fibers lie loosely on one another, which improves the absorbency and the flexibility of the layer 2 in these regions.

In the embodiment shown here, the further layer 3 is substantially thinner than the fibrous layer 2. The layer 3 can be formed, for example, from a web of textile, nonwoven or foil-like material.

The connection of the two layers 2, 3 takes place, for example, in an embossing method. This provides pressing in some regions, with compaction of the pulp fibers of the fibrous layer 2.

The pressing is not uniform over a large surface area, and instead pressing takes place mainly in embossed regions 4, wherein cup-shaped depressions 5 form in the fibrous layer 2. The depressions 5 in the embossed regions 4 can be round or polygonal. In the embodiment shown here, they have the shape of truncated pyramids or truncated cones.

The further layer 3 is only very thin, and therefore the embossed pattern resulting from the pressing is also visible on the outer face of the layer 3.

The embossing takes place by means of two rollers which, on their jacket surface, are provided with projections forming the depressions 5 and are basically embossing rollers. In this way, a compaction of the pulp fibers is obtained in the discrete embossed regions 4. In addition, a connection of the further layer 3 to the compacted embossed regions 4 takes place at least partially in these regions.

Outside the embossed region 4, the fibers lie loosely on one another, which improves the absorbency and ensures the flexibility of the material. In the production of the hygiene article according to this embodiment, neither adhesives nor other binders, e.g. latex, are needed. Instead, the connection is obtained by purely thermo-mechanical means. The specific arrangement, number and size of the embossed regions permits adjustment of the absorbency of the material.

The weight per unit area of the fibrous layer 2 can be between 15 g/m² and 600 g/m². The weight per unit area is preferably between 20 g/m² and 200 g/m². The weight per unit area of the further web 3 is generally slightly less than the weight per unit area of the fibrous layer 2 and is preferably between 15 and 30 g/m².

FIG. 2 shows a cross section through the material web 1 in the crimped state. The individual layers are not shown here. In the embodiment shown here, the material is arranged irregularly in folds. An absorption material for production of a hygiene article is obtained which, in addition to the embossed regions 4, has a surface structure with elevations and depressions. These elevations and depressions result in an enlarged surface area.

FIG. 3 shows a hygiene article according to the invention which has been formed from the crimped material web 1 shown in FIG. 2 and from a sheath 9, which has been partially omitted in FIG. 3. In the embodiment shown here, the material web 1 is folded in a zigzag shape, i.e. in alternating folds, which results in the formation of channels 11 extending in the longitudinal direction of the peg-shaped hygiene article. The alternating folding can be uniform or irregular. The liquid to be taken up can pass through the channels 11 and, via the liquid-permeable sheath 9, also through the fibrous layer of the material 1 and be absorbed. In the embodiment shown here, only the jacket surface is provided with a sheath. The end faces are uncovered, i.e. open.

In the embodiment shown here, the peg-shaped hygiene article is surrounded by a sheath 9. As has been described above, the sheath 9 should be configured such that adhesion of the hygiene article to the body surface is substantially avoided. However, it should have a certain porosity and permeability for the body fluid.

The surface 10 of the material 1 forms the inner surface of the channels 11. By means of the depressions 5, and by means of the optimal crimping of the material 1, an uneven surface structure of the channels 11 is formed which has a positive effect on the absorbency of the hygiene article according to the invention.

FIG. 4 shows a cross section through the hygiene article according to the invention along the line A-A. The material 1 is folded in a zigzag shape, i.e. in alternating folds, such that channels 11 form in the longitudinal direction of the hygiene article. The folded material 1 can be arranged symmetrically or asymmetrically inside the hygiene article 8.

In the embodiment shown here, the alternating folds of fibrous material 1 form individual circle segments 15 of a cylinder. However, the arrangement of the folds of the material 1 can also be irregular.

In a further embodiment, not shown here, the material web 1 can also be wound in the longitudinal direction.

The production of the hygiene articles according to the invention can in a manner known per se from the material 1. The production preferably takes place by so-called rod production. For this purpose, elongate, rod-shaped articles are first of all produced from the web material 1. This preliminary product is provided with the sheath 9. Web-shaped sheath materials can be wound. The sheath materials can also be used in the form of a tube and pulled over the rod-shaped preliminary product. Polymer films can be sprayed on as solutions or prepolymer and then dried or cured.

The hygiene article according to the invention can be configured in any desired sizes in order to be placed or inserted in a body cavity or opening so as to act as a plug.

### LIST OF REFERENCE SIGNS

1 web material

2 fibrous layer

3 further layer

4 embossed region

5 depressions

6 elevations

7 further depressions

8 hygiene article

9 sheath

10 surface of the web material 1

11 channels

12 end face

15 segment

1. A peg-shaped or cylindrical hygiene article for insertion into a body cavity or opening, which article comprises a fibrous layer (2) of pulp fibers which are interconnected in some regions by means of pressing, with the pulp fibers being compacted in the pressed regions (4), wherein the fibrous layer (2) is in the form of a fibrous web that is folded so as to form channels (11) extending in the longitudinal direction of the hygiene article.

2. The hygiene article as claimed in claim 1, characterized in that the jacket surface is provided at least partially with a liquid-permeable sheath (9).

3. The hygiene article as claimed in claim 2, characterized in that the sheath (9) is a nonwoven, a net, tissue, foil or polymer film.

4. The hygiene article as claimed in claim 1, characterized in that the pulp fibers in the pressed regions (4) are pressed by exposure to heat.

5. The hygiene article as claimed in claim 1, characterized in that neither adhesives nor other binders are used in the production of the fibrous web.

6. The hygiene article as claimed in claim 1, characterized in that the fibrous layer (2) has a further layer (3) on one flat side.

7. The hygiene article as claimed in claim 6, characterized in that the fibrous layer has further layers (3) on both flat sides, wherein the further layers can be made from the same material or from materials different from each other.

8. The hygiene article as claimed in claim 7, characterized in that each of the further layers (3) is chosen independently of each other from tissue, textile, nonwoven or foil-like material.

9. The hygiene article as claimed in claim 1, characterized in that the fibrous layer has a weight per unit area of between 15 g/m² and 600 g/m², preferably between 20 g/m² and 200 g/m².

10. The hygiene article as claimed in claim 1, characterized in that it contains one or more additives chosen from absorbents and/or further auxiliaries.

11. The hygiene article as claimed in claim 10, characterized in that the additives are chosen from absorbents, fragrances, flavorings, aromas and/or pharmaceutical active substances.

12. The hygiene article as claimed in claim 11, characterized in that the additives are chosen from superabsorbent polymers, activated carbons, zeolites, silicates, SiO₂, disinfecting or hemostatic substances, surface-active anesthetics, menthol, clove oil, cocoa, vanilla and any desired mixtures of the above.
